(19) United States
(12) Patent Application Publication
(10) Pub. No.: US 2012/0202700 A1
(43) Pub. Date: Aug. 9, 2012

(54) Sample preparation and detection method

(76) Inventors: Raymond M. Pierson, Elkridge, MD (US); Matthew W. Snyder, Elkridge, MD (US); Karen G. Jarvis, Elkridge, MD (US)

(21) Appl. No.: 12/292,426

(22) Filed: Nov. 19, 2008

(51) Int. Cl. C12Q 1/68; C40B 30/04

(52) U.S. Cl. 506/9; 435/6.12

(57) ABSTRACT

A method for detecting a biological agent in a liquid sample is disclosed. The method comprises: passing a liquid sample through a filter in the presence of a surfactant; and subjecting the filtered sample to direct polymerase chain reaction (PCR) analysis for the presence of a biological agent, wherein the filter has a porosity that allows the biological agent to pass through the filter in its intact form.

### TECHNICAL FIELD

The embodiments described herein relate generally to sample preparation and detection methods, and more particularly, to a method for preparing samples for PCR analysis using filtration in the presence of a surfactant.

### BACKGROUND

In recent years, there has been demand for methods and devices for detecting biological agents that may be used in a terrorism attack. The first step of the detection process is to collect samples that may contain a biological agent. The collected samples are then analyzed for the presence of a biological agent or agents. Due to the diverse nature of the sample collection methods, the collected samples often contains a wide variety of “background” materials that must be removed before the analysis step. The process is often referred to as the “sample preparation step.” There is a need for a sample preparation method that is simple and efficient, and is capable of delivering high quality samples for further analysis.

### SUMMARY

A method for detecting a biological agent in a liquid sample is disclosed. The method comprises: passing a liquid sample through a filter in the presence of a surfactant; and subjecting the filtered sample to direct polymerase chain reaction (PCR) analysis for the presence of a biological agent, wherein the filter has a porosity that allows the biological agent to pass through the filter in its intact form.

Also disclosed is a method for collecting and detecting a biological agent. The method comprises: collecting particles from a fluid sample; suspending the collected particles in a liquid to form a concentrated liquid sample; passing the concentrated liquid sample through a filter in the presence of a surfactant to produce a filtered sample, wherein the filter has a porosity that allows the biological agent to pass through said filter in its intact form; and analyzing the filtered sample for the presence of the biological agent.

### DETAILED DESCRIPTION OF DRAWINGS

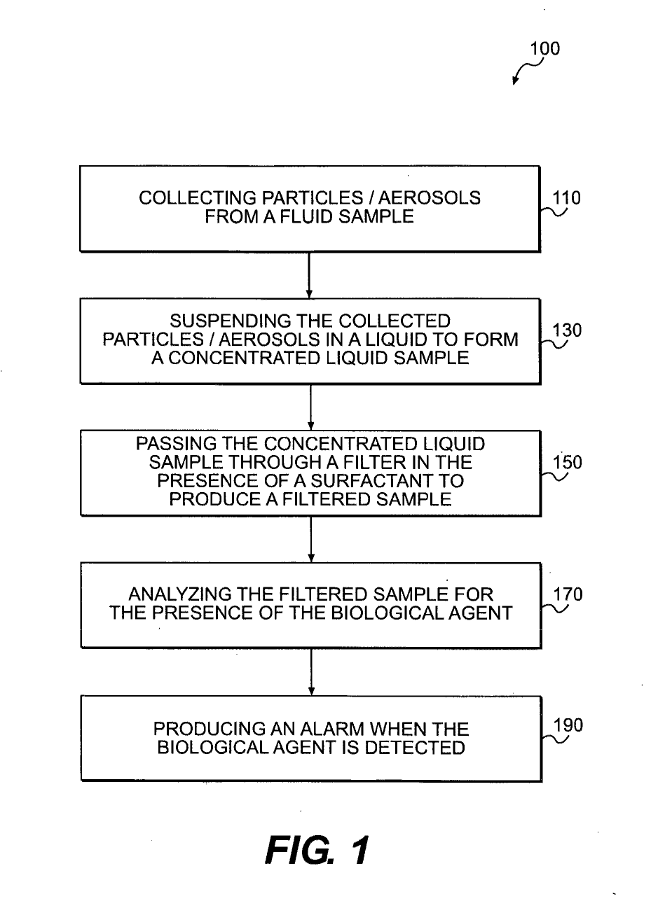

FIG. 1 is a flow chart showing an embodiment of a method of sample preparation and analysis.

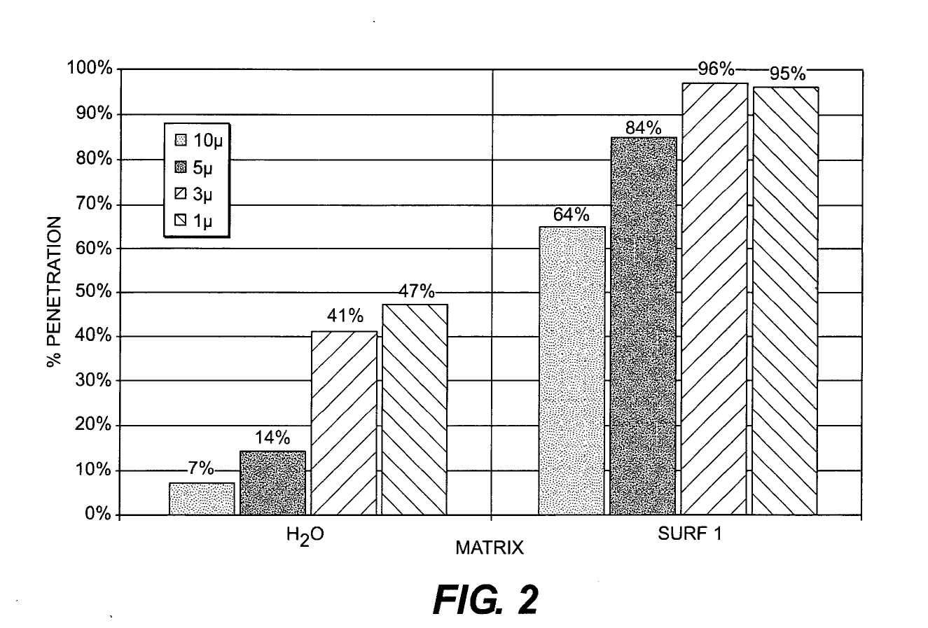

FIG. 2 is a diagram showing penetration of polystyrene latex (PSL) particles across a polyproprolene filter.

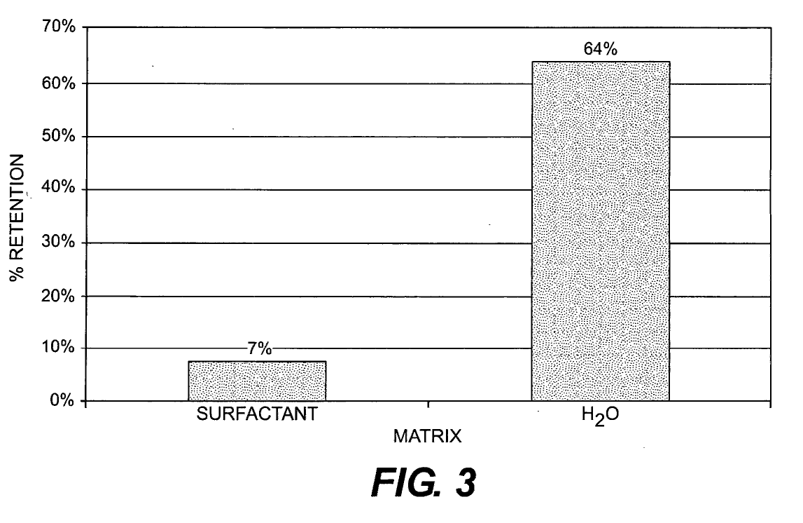

FIG. 3 is a diagram showing retention of Visolite® particles on a polyproprolene filter.

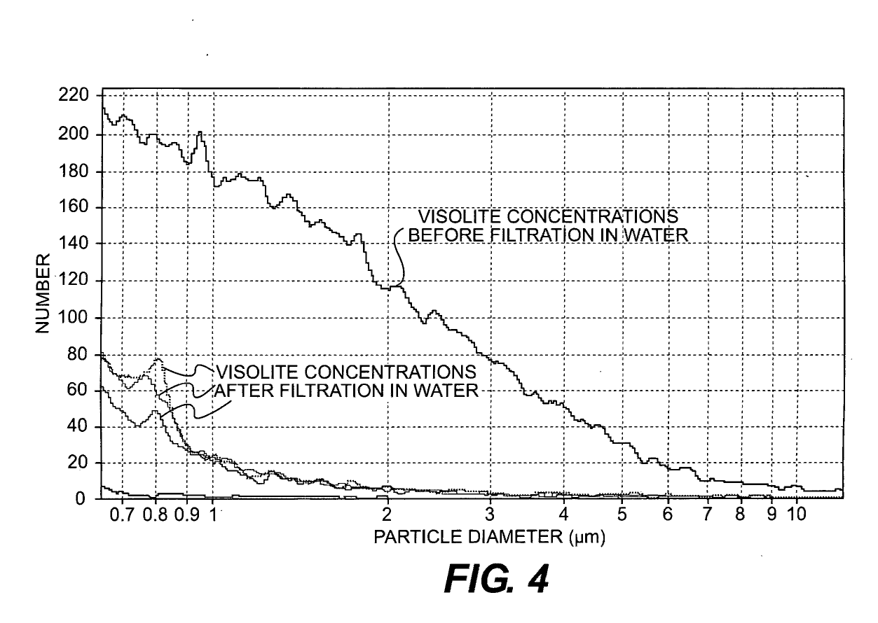

FIG. 4 is a diagram showing filtration efficiency of Visolite® particles in the absence of surfactant.

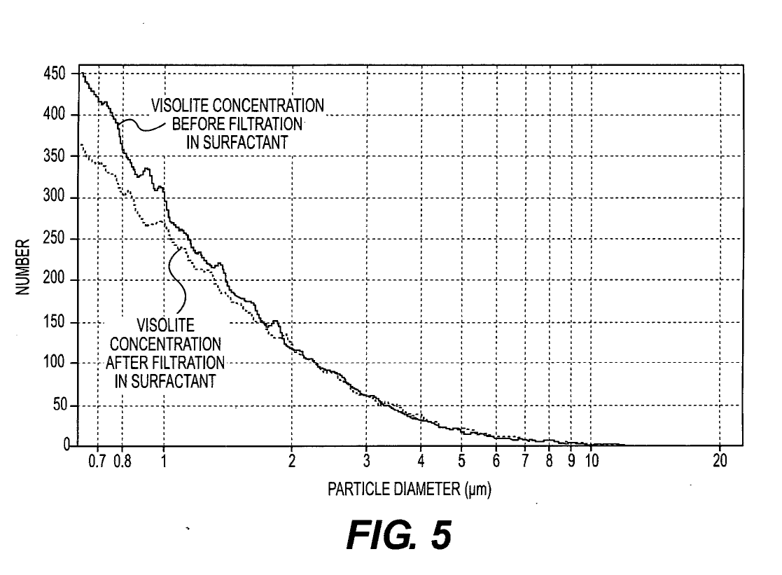

FIG. 5 is a diagram showing filtration efficiency of Visolite® particles in the presence of surfactant.

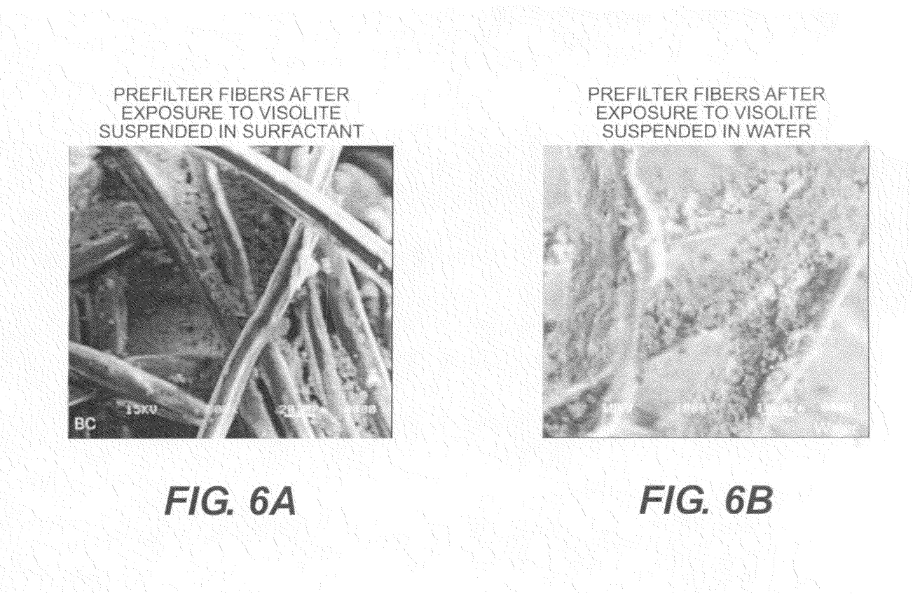

FIGS. 6A and 6B are electron microscope pictures of the filter material after exposure to Visolite® particles suspended in water with surfactant (FIG. 6A) and Visolite® particles suspended in water with surfactant (FIG. 6B).

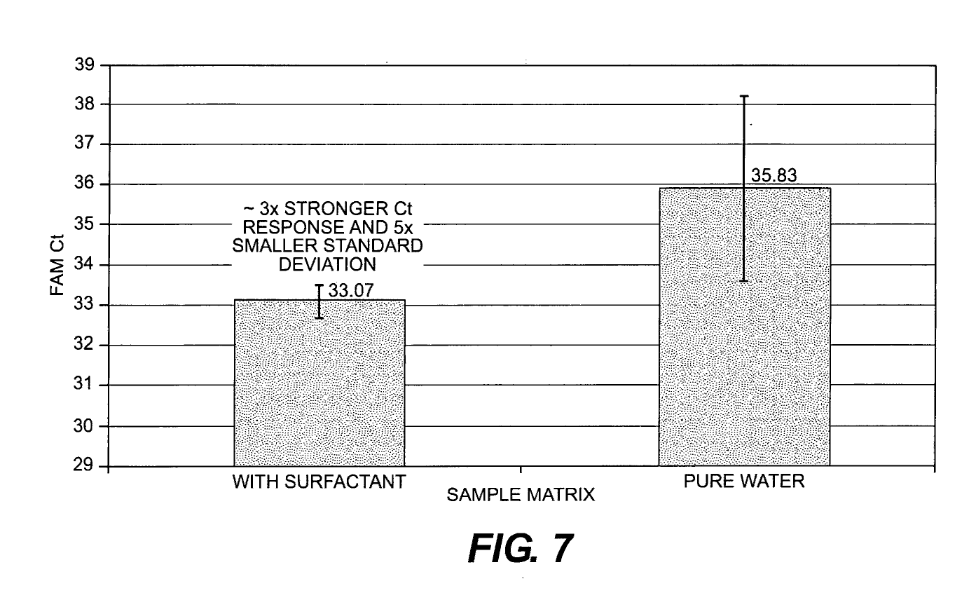

FIG. 7 is a diagram showing PCR response to Gamma killed Bacillus Anthracis spores filtered in the presence or absence of surfactant.

### DETAILED DESCRIPTION

Described herein is a sample preparation method for the detection of biological agents. The terms and descriptions used herein are set forth by way of illustration only and are not meant as limitations. Those skilled in the art will recognize that many variations are possible within the spirit and scope of the invention as defined in the following claims, and their equivalents, in which all terms are to be understood in their broadest possible sense unless otherwise indicated.

Referring now to FIG. 1, an embodiment of the method 100 for collecting and detecting a biological agent includes collecting (110) particles/aerosols from a fluid sample; suspending (130) the collected particles/aerosols in a liquid to form a concentrated liquid sample; passing (150) the concentrated liquid sample through a filter in the presence of a surfactant to produce a filtered sample; analyzing (170) the filtered sample for the presence of the biological agent, and producing (190) an alarm when the biological agent is detected.

The biological agent can be any microorganism of interest. Examples of the microorganisms of interest include, but are not limited to, eukaryotic and prokaryotic cells, parasites, bacteria, virus particles and prions. Examples of eukaryotic cells include all types of animal cells, such as mammal cells, reptile cells, amphibian cells, and avian cells, blood cells, hepatic cells, kidney cells, skin cells, brain cells, bone cells, nerve cells, immune cells, lymphatic cells, brain cells, plant cells, and fungal cells. In another aspect, the biological agent can be a component of a cell including, but not limited to, the nucleus, the nuclear membrane, leucoplasts, the microtrabecular lattice, endoplasmic reticulum, ribosomes, chromosomes, cell membrane, mitochondrion, nucleoli, lysosomes, the Golgi bodies, peroxisomes, or chloroplasts.

Examples of bacteria include, but are not limited to, Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus, Alteromonas, Amycolata, Amycolatopsis, Anaerobospirillum, Anaerorhabdus, Arachnia, Arcanobacterium, Arcobacter, Arthrobacter, Atopobium, Aureobacterium, Bacteroides, Balneatrix, Bartonella, Bergeyella, Bifidobacterium, Bilophila Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Francisella, Fusobacterium, Gardnerella, Gemella, Globicatella, Gordona, Haemophilus, Hafnia, Helicobacter, Helococcus, Holdemania Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, Oeskovia, Oligella, Orientia, Paenibacillus, Pantoea, Parachlamydia, Pasteurella, Pediococcus, Peptococcus, Peptostreptococcus, Photobacterium, Photorhabdus, Plesiomonas, Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia Rochalimaea Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia, and Yokenella. Other examples of bacterium include Mycobacterium tuberculosis, M. bovis, M. typhimurium, M. bovis strain BCG, BCG substrains, M. avium, M. intracellulare, M. africanum, M. kansasii, M. marinum, M. ulcerans, M. avium subspecies paratuberculosis, Staphylococcus aureus, Staphylococcus epidermidis, Staphylococcus equi, Streptococcus pyogenes, Streptococcus agalactiae, Listeria monocytogenes, Listeria ivanovii, Bacillus anthracis, B. subtilis, Nocardia asteroides, and other Nocardia species, Streptococcus viridans group, Peptococcus species, Peptostreptococcus species, Actinomyces israelii and other Actinomyces species, and Propionibacterium acnes, Clostridium tetani, Clostridium botulinum, other Clostridium species, Pseudomonas aeruginosa, other Pseudomonas species, Campylobacter species, Vibrio cholerae, Ehrlichia species, Actinobacillus pleuropneumoniae, Pasteurella haemolytica, Pasteurella multocida, other Pasteurella species, Legionella pneumophila, other Legionella species, Salmonella typhi, other Salmonella species, Shigella species Brucella abortus, other Brucella species, Chlamydi trachomatis, Chlamydia psittaci, Coxiella burnetti, Escherichia coli, Neiserria meningitidis, Neiserria gonorrhea, Haemophilus influenzae, Haemophilus ducreyi, other Hemophilus species, Yersinia pestis, Yersinia enterolitica, other Yersinia species, Escherichia coli, E. hirae and other Escherichia species, as well as other Enterobacteria, Brucella abortus and other Brucella species, Burkholderia cepacia, Burkholderia pseudomallei, Francisella tularensis, Bacteroides fragilis, Fudobascterium nucleatum, Provetella species, and Cowdria ruminantium, or any strain or variant thereof.

Examples of viruses include, but are not limited to, Herpes simplex virus type-1, Herpes simplex virus type-2, Cytomegalovirus, Epstein-Barr virus, Varicella-zoster virus, Human herpesvirus 6, Human herpesvirus 7, Human herpesvirus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-I, Hantavirus, Rubella virus, Simian Immunodeficiency virus, Human Immunodeficiency virus type-1, Vaccinia virus, SARS virus, and Human Immunodeficiency virus type-2, or any strain or variant thereof.

Examples of parasites include, but are not limited to, Toxoplasma gondii, Plasmodium falciparum, Plasmodium vivax, Plasmodium malariae, other Plasmodium species, Trypanosoma brucei, Trypanosoma cruzi, Leishmania major, other Leishmania species, Schistosoma mansoni, other Schistosoma species, and Entamoeba histolytica, or any strain or variant thereof.

The fluid sample may be virtually any fluid suspected of containing a biological agent of interest. The term “fluid,” as use in the embodiments described herein, refers to a substance that continually deforms (flows) under an applied shear stress regardless of how small the applied stress. Exemplary fluid samples include air samples and liquid samples. Examples of liquid samples include, but are not limited to, water samples, wash liquids from foods or food processing equipment, milk, fruit and vegetable juices, blood, plasma, urine, and solid materials suspended in a liquid.

The particles/aerosols in the fluid sample may be collected (110) using conventional particles collection methods. In one embodiment, the particles/aerosols are collected using a commercial off-the-shelf particle/aerosol collector. Examples of the particle/aerosol collectors include, but are not limited to, electrostatic collectors, virtual impactors, regular plate impactors, cyclone collectors and filter-based collectors. The collection conditions, such as the sample flow rate and collecting temperature, may be optimized for the biological agent of interest.

In one embodiment, the particles/aerosols are collected (110) with an electrostatic collector. The electrostatic collector removes particles from an air sample by using electrostatics to direct the particles or aerosols onto a metal grid or into a liquid, creating a highly concentrated particle/aerosol sample.

In another embodiment, the particles/aerosols are collected (110) with a virtual impactor with a desired threshold size. Briefly, a jet of particle-laden air is accelerated toward a collection probe positioned downstream so that a small gap exists between the acceleration nozzle and the probe. A vacuum is applied to deflect a major portion of the airstream through the small gap. Particles larger than a preset threshold size, known as the cutpoint, have sufficient momentum so that they cross the deflected streamlines and enter the collection probe, whereas smaller particles follow the deflected airstream. Larger particles are removed from the collection probe by the minor portion of the airstream according to the magnitude of the vacuum applied to the minor portion.

In another embodiment, the particles/aerosols are collected (110) with a regular impactor. The particles are accelerated through a nozzle towards an impactor plate maintained at a fixed distance from the nozzle. The plate deflects the flow creating fluid streamlines around itself. Due to inertia, the larger particles are impacted (and collected) on a collector plate while the smaller particles follow the deflected streamlines.

In another embodiment, the particles/aerosols are collected (110) with cyclones or centrifugal collectors that create a ‘cyclonic’ or centrifugal force to separate particles/aerosols from a fluid sample stream. The centrifugal force is created when the fluid sample enters the top of the collector at an angle and is spun rapidly downward in a vortex (similar to a whirlpool action). As the fluid sample flow moves in a circular fashion downward, heavier particles are thrown against the walls of the collector, collect, and slide down into a hopper.

In yet another embodiment, the particles/aerosols are collected (110) with a filter-based collector that collects particles/aerosols on a filter. The filter can be a porous material that traps particles/aerosols.

The collected particles/aerosols are then suspended (130) in a suspension liquid to form a concentrated liquid sample. Typically, the concentrated liquid sample contains particles of various sizes and a wide variety of “background” materials that need to be removed to ensure the reliability of the down-stream analysis. Particles that are larger than the particles of interest are removed by filtration. For example, most viruses and bacteria have a size of less than 10 microns. Therefore, if the particles of interest are such biowarfare agents, it is possible to pass the concentrated liquid sample through a 10 micron filter to remove larger particles that are not of interest.

However, most biological particles are charged particles that tend to be retained by the filter regardless of their physical size. In addition, the electrical charges on biological particles may change randomly, causing fluctuation in the filtration efficiency and a high degree of variability in the final analysis. In the embodiments described herein, the concentrated liquid sample is filtered (150) in the presence of a surfactant to produce a filtered sample. The filtrating step isolates the biological particles of interest from larger particles or other objects in the collected sample. As used in the embodiments described herein, “isolating” occurs when one or more of the particles of interest are substantially separated from other larger components of the sample. When the particle is an organism, one or more different types of organisms may be together in the product of the isolation, and the isolated organism may be viable or non-viable.

As used herein, the term “surfactant” is intended to mean a substance that functions to reduce the surface tension of a liquid in which it is dissolved. Surfactants include, for example, amphipathic organic compounds that exhibit partial solubility in both organic solvents and aqueous solutions. General characteristics of surfactants include their ability to reduce the surface tension of water, reduce the interfacial tension between oil and water and also form micelles. Surfactants of the invention include non-ionic and ionic surfactants. Surfactants are well known in the art and can be found described in, for example, Randolph T. W. and Jones L. S., Surfactant-protein interactions. Pharm Biotechnol. 13:159-75 (2002).

Examples of non-ionic surfactants include, but are not limited to, alkyl poly (ethylene oxide), alkyl polyglucosides such as octyl glucoside and decyl maltoside, fatty alcohols such as cetyl alcohol and oleyl alcohol, cocamide MEA, cocamide DEA, and cocamide TEA. Specific examples of non-ionic surfactants include the polysorbates including, for example, polysorbate 20, polysorbate 28, polysorbate 40, polysorbate 60, polysorbate 65, polysorbate 80, polysorbate 81, polysorbate 85 and the like; the poloxamers including, for example, poloxamer 188, also known as poloxalkol or poly(ethylene oxide)-poly(propylene oxide), poloxamer 407 or polyethylene-polypropylene glycol and the like, and polyethylene glycol (PEG).

Ionic surfactants include anionic, cationic and zwitterionic surfactants. Examples of anionic surfactants include, but are not limited to, sulfonate-based or carboxylate-based surfactants such as soaps, fatty acid salts, sodium dodecyl sulfate (SDS), ammonium lauryl sulfate and other alkyl sulfate salts. Examples of cationic surfactants include, but are not limited to, quaternary ammonium-based surfactants such as cetyl trimethylammonium bromide (CTAB), other alkyltrimethylammonium salts, cetyl pyridinium chloride, polyethoxylated tallow amine (POEA) and benzalkonium chloride. Examples of zwitterionic or amphoteric surfactants include, but are not limited to, dodecyl betaine, dodecyl dimethylamine oxide, cocamidopropyl betaine and coco ampho glycinate.

The type and amount of surfactant is application-dependent. In one embodiment, the surfactant is a non-ionic surfactant. In another embodiment, the surfactant is an ionic surfactant. In another embodiment, the surfactant is SDS. In another embodiment, the surfactant is Dynasolve™ (Dynaloy Indianapolis, Ind.). In another embodiment, the surfactant is PEG. In another embodiment, the surfactant is used in an amount within the range of 0.01-2% (w/w). In another embodiment, the surfactant is used in an amount within the range of 0.1-1% (w/w).

In some embodiments, samples are filtered (150) without the collection step (110) and the re-suspension step (130). In one embodiment, a liquid sample is filtered (150) in the presence of a surfactant to produce a filtered sample, which is then analyzed for the presence of a biological agent by PCR. Samples with a simple makeup, such as a water sample, may be filtered directly. Samples with more complex makeup, such as foods, tissue samples, or other biologically-derived materials, however, may subject to several processing steps to allow adequate recovery of isolated organisms. This is particularly true in instances when the isolated organism is to be detected with equipment that is sensitive to impurities, such as a biochip.

In one embodiment, a sample is processed to create a bioagent-containing liquid component and this liquid component is then separated from non-liquid material. Processing steps can include dilution, blending, chopping, centrifugation, filtrations such as vacuum filtration through various depth filters and filter aid facilitated filtration, processing through rolled stationary phase, enzyme treatment (e.g., lipases, proteases, amylases), lipid extraction (e.g., with ethanol, methanol, and/or hexane), massaging, and contacting the solution with positively-charged or negatively-charged membrane materials or particles. A single processing step may be used one or more times or two or more processing steps may be used in combination.

In another embodiment, an additional purification step is performed prior to, or after the filtration (150). The additional purification step may be performed using purification technologies well known in the art, such as centrifugation or affinity purification. The additional purification step may also be another filtration step.

Next, the filtered sample is analyzed (170) for the presence of the biological agent of interest. In one embodiment, the filtered sample is subjected to polymerase chain reaction (PCR) analysis for the presence of the biological agent of interest. In another embodiment, the filtered sample is analyzed with a biochip. The term “biochip” as used herein, refers to a collection of miniaturized test sites (microarrays) arranged on a solid substrate that permits many tests to be performed at the same time in order to achieve higher throughput and speed. Examples of the microarrays include, but are not limited to, nucleotide microarrays, protein microarrays, and antibody microarrays.

### EXAMPLES

The following specific examples are intended to illustrate the collection and detection of representative biological agents using methods described in the embodiments. The examples should not be construed as limiting the scope of the claims.

### Example 1

### Filtration of Polystyrene Latex (PSL) Particles with Polyproprolene Filters

Hydrophobic, charge neutral PSL particles in sizes ranging from 1 to 10 μm are suspended in either water or water with a sub-percent level of surfactant, such as sodium dodecyl sulphate (SDS). The particle suspensions are filtered with a polyproprolene filter having a physical 50% cut point of 10 μm. As shown in FIG. 2, particles suspended in water with surfactant (Surf 1) have a much higher penetration rate (i.e., the percentage of particles that pass the filter) than particles suspended in water without surfactant (H₂O).

### Example 2

### Filtration of Visolite Particles with Polyproprolene Filters

Since PSL particles carry only a small negative charge, they are not considered the best simulant material for anthracis spores. Experiment 1 is repeated using Visolite® powders (GE Energy, Kansas City, Mo.) as a simulant to anthracis spores. Visolite® particles are hydrophobic and have a negative charge that is closer to that of the anthracis spores. The particles are polydispersed in sized from 0.6 to 12 μm with a mean diameter of 1.6 μm (the approximate size of anthracis spores). As shown in FIG. 3, the presence of surfactant significantly reduces the retention rate from 64% to 7%.

The pre- and post-filtration Visolite® suspensions are also analyzed with a Beckman Coulter Multisizer. As shown in FIG. 4, in the absence of surfactant, there is a significant drop in Visolite® concentration after filtration, suggesting that a large fraction of the Visolite® particles are retained by the filter material. In contrast, the pre- and post-filtration Visolite® concentration are similar in the presence of surfactant (FIG. 5), indicating a low retention rate by the filter material. Electron microscope analysis further confirmed that the presence of surfactant significantly reduces the retention of Visolite® particles by polyproprolene filters. As shown in FIG. 6A, there is little accumulation of Visolite® particles in the filter after passing a surfactant-containing Visolite® suspension through the filter. FIG. 6B shows the heavy accumulation of Visolite® particles in the filter after passing a Visolite® suspension through the filter in the absence of surfactant.

### Example 3

### PCR Detection of Filtered Bacillus anthracis Spores

Gamma killed Bacillus Anthracis spores are suspended in pure water or water containing 0.5% sodium dodecyl sulphate (SDS). The suspensions are filtered with a polyproprolene filter having a physical 50% cut point of 10 μm. The filtered suspensions are then analysis by real-time PCR with primers specific to Bacillus Anthracis. Compared to samples filtered in pure water, samples filtered in the present of SDS showed stronger (about 10×) Ct response and smaller (about ⅕) standard deviation (FIG. 7). These results are consistent with the results in Examples 1 and 2.

The foregoing discussion discloses and describes many exemplary methods and embodiments of the present invention. As will be understood by those familiar with the art, the invention may be embodied in other specific forms without departing from the spirit or essential characteristics thereof. Accordingly, the disclosure of the present invention is intended to be illustrative, but not limiting, of the scope of the invention, which is set forth in the following claims.

What is claimed is:

1. A method for detecting a biological agent in a liquid sample, comprising:
passing said liquid sample through a filter in the presence of a surfactant; and
subjecting the filtered sample to direct polymerase chain reaction (PCR) analysis for the presence of said biological agent,
wherein said filter has a porosity that allows said biological agent to pass through said filter in its intact form.

2. The method of claim 1, wherein said surfactant is an ionic surfactant.

3. The method of claim 2, wherein said surfactant is sodium dodecyl sulphate.

4. The method of claim 1, wherein said surfactant is Dynasolve.

5. The method of claim 1, wherein said surfactant is polyethylene glycol.

6. The method of claim 1, wherein said surfactant is present in the concentration range of 0.01-2% (w/w).

7. The method of claim 1, wherein said surfactant is present in the concentration range of 0.1-1% (w/w).

8. The method of claim 1, wherein said surfactant is present at a concentration of about 0.5% (w/w).

9. The method of claim 1, wherein said filter is a polyproprolene filter.

10. The method of claim 1, further comprising the step of producing an alarm when said biological agent is detected.

11. The method for collecting and detecting a biological agent, comprising:
collecting particles from a fluid sample;
suspending the collected particles in a liquid to form a concentrated liquid sample;
passing said concentrated liquid sample through a filter in the presence of a surfactant to produce a filtered sample, wherein said filter has a porosity that allows said biological agent to pass through said filter in its intact form, and
analyzing the filtered sample for the presence of said biological agent.

12. The method of claim 11, wherein said particles are collected using a collector selected from the group consisting of electrostatic collectors, virtual impactors, regular plate impactors, cyclone collectors and filter-based collectors.

13. The method of claim 11, wherein said surfactant is selected from the group consisting of sodium dodecyl sulphate, Dynasolve™, and polyethylene glycol

14. The method of claim 11, wherein said surfactant is present in the concentration range of 0.01-2% (w/w).

15. The method of claim 11, wherein said surfactant is present in the concentration range of 0.1-1% (w/w).

16. The method of claim 11, wherein said surfactant is sodium dodecyl sulphate and is present at a concentration of about 0.5% (w/w).

17. The method of claim 11, wherein said filter is a polyproprolene filter.

18. The method of claim 11, further comprising the step of producing an alarm when said biological agent is detected.

19. The method of claim 11, wherein the filtered sample is analyzed by PCR analysis.

20. The method of claim 11, wherein the filtered sample is analyzed with a biochip.
